(19) United States
(12) Patent Application Publication
(10) Pub. No.: US 2018/0085227 A1
(43) Pub. Date: Mar. 29, 2018

(54) TOTAL ANKLE REPLACEMENT JOINT

(71) Applicant: Articulus Bio, LLC, Boulder, CO (US)

(72) Inventor: Jerome Rifkin, Louisville, CO (US)

(21) Appl. No.: 15/714,914

(22) Filed: Sep. 25, 2017

Related U.S. Application Data
(60) Provisional application No. 62/399,063, filed on Sep. 23, 2016.

(51) Int. Cl. A61F 2/42

(52) U.S. Cl. CPC A61F 2/4202; A61F 2002/30754; A61F 2002/4207; A61F 2002/4205

(57) ABSTRACT

In a total ankle replacement joint, tibial and talar components are coupled by first and second tension members threaded through the tibial component and the talar component to support the tibial component relative to the talar component. The total ankle replacement joint is configured such that the tibial component rocks relative to the talar component on fixed-length web member segments of the first and second tension members and fixed-length loop member segments of the first and second tension members constrain motion of the tibial component relative to the talar component.

### CROSS-REFERENCE TO RELATED APPLICATIONS

This application claims the priority benefit of U.S. Provisional Application No. 62/399,063 filed on Sep. 23, 2016 which is incorporated herein by reference in its entirety.

### SUMMARY

The present disclosure seeks to provide a total ankle replacement joint, including a tibial component, a talar component and first and second tension members threaded through the tibial component and the talar component to couple the tibial component with the talar component and support the tibial component relative to the talar component.

The present disclosure also seeks to provide an implant device for total joint replacement, the apparatus including a first implantable articulation component having a first end and a second end, the first end configured for implantation to bone forming a first side of a joint being replaced by the implant device, having a second implantable articulation component having a first end and a second end, the first end configured for implantation to bone forming a second side of the joint being replaced by the implanted device and including at least one tension member extending between the first implantable articulation component and the second implantable articulation component, wherein the at least one tension member is configured to dispose the second end of the first implantable articulation component and the second end of the second implantable articulation component toward one another, and the at least one tension member is configured to allow a given amount of articulation of the first implantable articulation component and the second implantable articulation component with respect to one another.

The present disclosure further seeks to provide an implant device for total joint replacement, including a tibial component having first and second peripheral anchors and at least one intermediate anchor, a talar component having first and second peripheral anchors and at least one intermediate anchor, a first tension member threaded through the first peripheral anchor of the tibial component and the first peripheral anchor of the talar component and configured to constrain motion of the first peripheral anchor of the tibial component to a fixed radius relative to the first peripheral anchor of the talar component, a second tension member threaded through the second peripheral anchor of the tibial component and the second peripheral anchor of the talar component and configured to constrain motion of the second peripheral anchor of the tibial component about a fixed radius relative to the second peripheral anchor of the talar component and a third tension member threaded through the at least one intermediate anchor of the tibial component and the at least one intermediate anchor of the talar component and configured to constrain motion of the at least one intermediate anchor of the tibial component to a fixed radius relative to the intermediate anchor of the talar component.

### BRIEF DESCRIPTION OF THE FIGURES

The summary above, as well as the following detailed description of illustrative embodiments, is better understood when read in conjunction with the appended drawings. For the purpose of illustrating the present disclosure, example constructions of the disclosure are shown in the drawings. However, the present disclosure is not limited to specific methods and instrumentalities disclosed herein. Moreover, those in the art will understand that the drawings are not to scale. Wherever possible, like elements have been indicated by identical numbers.

Embodiments of the present disclosure will now be described, by way of example only, with reference to the following diagrams wherein:

FIG. 1 illustrates a perspective view of an example total ankle replacement in accordance with the disclosure.

FIG. 2 illustrates a front or anterior view of an example total ankle replacement in accordance with the disclosure.

FIG. 3 illustrates a right side or medial view of an example total ankle replacement in accordance with the disclosure.

FIG. 4 illustrates a rear or posterior view of an example total ankle replacement in accordance with the disclosure.

FIG. 5 illustrates a left side or lateral view of an example total ankle replacement in accordance with the disclosure.

FIG. 6 illustrates a top or superior view of an example total ankle replacement in accordance with the disclosure.

FIG. 7 illustrates an exploded perspective view of an example total ankle replacement in accordance with the disclosure.

FIG. 8 illustrates a top or superior view of an example total ankle replacement in accordance with the disclosure.

FIG. 9A illustrates a large scale right side or medial view showing section line A-A.

FIG. 9B illustrates a sectional view through line A-A of FIG. 9A.

FIGS. 10A-F illustrate six views of an example tibial component of an example total ankle replacement in accordance with the disclosure.

FIGS. 11A-E illustrate five views of an example first, upper, or superior wafer of an example talar component in accordance with the disclosure.

FIGS. 12A-E illustrate five views of an example second or intermediate wafer of an example talar component in accordance with the disclosure.

FIGS. 13A-E illustrate five views of an example third, lower or inferior wafer of an example talar component in accordance with the disclosure.

FIGS. 14A-E illustrate five views of an example talar base plate of an example total ankle replacement in accordance with the disclosure.

FIGS. 15A-C illustrate three views of an example tibial base plate of an example total ankle replacement in accordance with the disclosure.

FIG. 16 illustrates an example simplified total ankle replacement joint.

FIG. 17 illustrates a front or anterior view of an example installation of an example total ankle replacement in accordance with the disclosure.

### DETAILED DESCRIPTION

The following detailed description illustrates embodiments of the present disclosure and manners by which they can be implemented. Although the best mode of carrying out the present disclosure has been disclosed, those skilled in the art would recognize that other embodiments for carrying out or practicing the present disclosure are also possible.

It should be noted that the terms “first”, “second”, and the like, herein do not denote any order, quantity, or importance, but rather are used to distinguish one element from another. Further, the terms “a” and “an” herein do not denote a limitation of quantity, but rather denote the presence of at least one of the referenced item.

A variety of joints are known however most employ very old technology such as pin joints and spherical joints. Joints usable as implants are typically arranged as sliding joints having metal and ultra-high molecular weight polyethylene (UHMWPE) bearing surfaces. Disadvantageously, the relative sliding of these bearing surfaces causes the generation of polyethylene or metal debris which migrate into surrounding tissue potentially resulting in infection and bone loss which necessitate a major revision surgery.

In particular, known total ankle replacement joints employ a saddle-shaped joint between UHMWPE and metal surfaces. When a patient implanted with one of these known joints executes axial rotation around their tibial shaft while standing, their foot does rotate relative to the tibial component. Instead, the patient's body weight is lifted up the inclined plane of the saddle-shaped joint. The torque required for this lifting is then transmitted down to the talar bone/metal interface potentially causing damage to the bone/metal interface. This is more of a problem in the talus than the tibia, because the talus has poor blood supply and is more prone to avascular necrosis and pressure related subsidence.

Embodiments of the present disclosure substantially eliminate, or at least partially address, problems in the prior art. By using wrapping tendon webjoints, disclosed joints avoid relative sliding of bearing surfaces and the resultant wear debris. Because disclosed joints experience minimal height gain during axial rotation, high torque and increased loading of the metal/bone interfaces are avoided.

Disclosed tension members create joints with predictable, tunable ranges of motion in accordance with geometry. Like quasi-stable tensegrities in tensegrity joints, disclosed compression members provide a shape to a web of tension members. By altering the shape of the compression members, joints can be created with more or less movement allowed in some directions. Compliant joints with indefinite centers of motion are disclosed which allow for more efficient motions which are similar to anatomic systems.

Additional aspects, advantages, features and objects of the present disclosure will be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

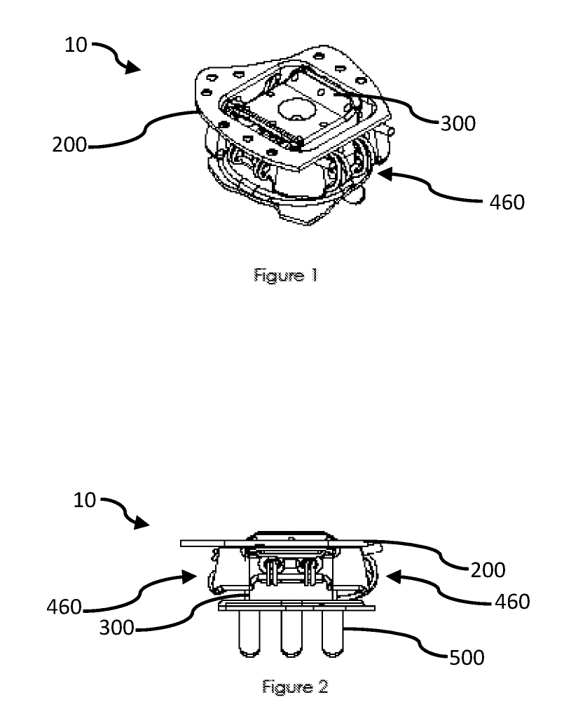

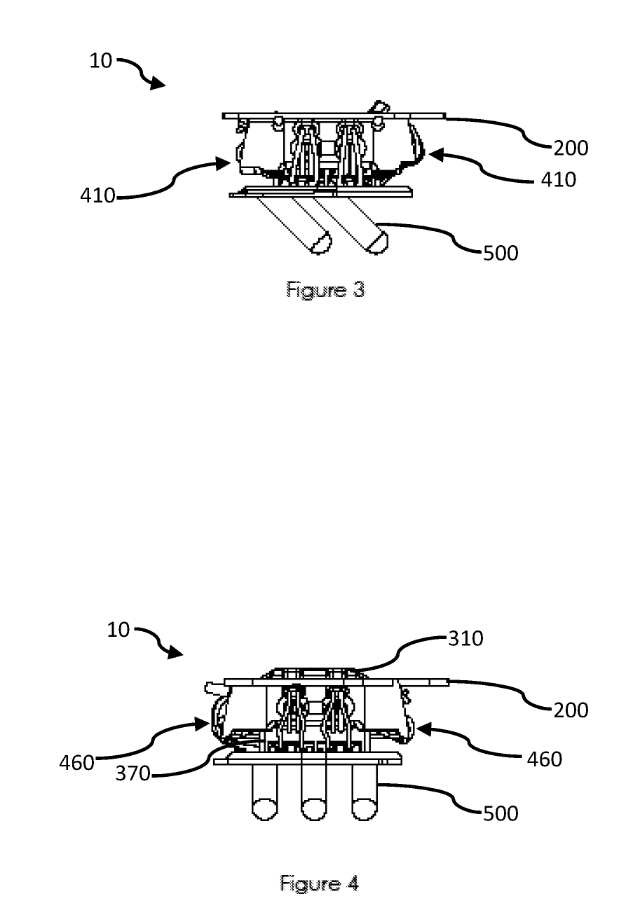

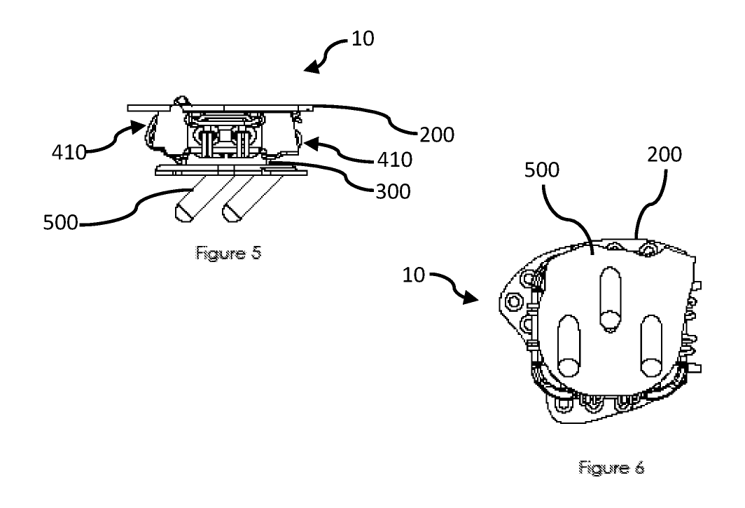

Referring to FIGS. 1-6, a total ankle replacement joint 10 includes two compression members: a tibial component 200 configured for fixing to a tibia and a talar component 300 configured for fixing to a talus. Tibial component 200 and talar component 300 provide shape for first 410 and second 460 tension members which are threaded therethrough to couple tibial component 200 with talar component 300. First and second tension members 410 and 460 provide movement and strength to the joint and support tibial component 200 relative to talar component 300. Talar component 300 is configured to be received within four walls of tibial component 200. While tibial component 200 and talar component 300 may be thought of as articulation members, the first 410 and second 460 tension members substantially prevent contact between tibial component 200 and talar component 300 so tibial component 200 does not articulate directly on talar component 300.

Tibial component 200 further comprises a plurality of walls which are each independently supported by at least one segment of the first tension member 410 or at least one segment of the second tension member 460. The total ankle replacement joint is configured such that tibial component 200 rocks relative to the talar component on the first and second tension members 410 and 460. The first and second tension members 410 and 460 constrain motion of tibial component 200 relative to talar component 300 about a moving center.

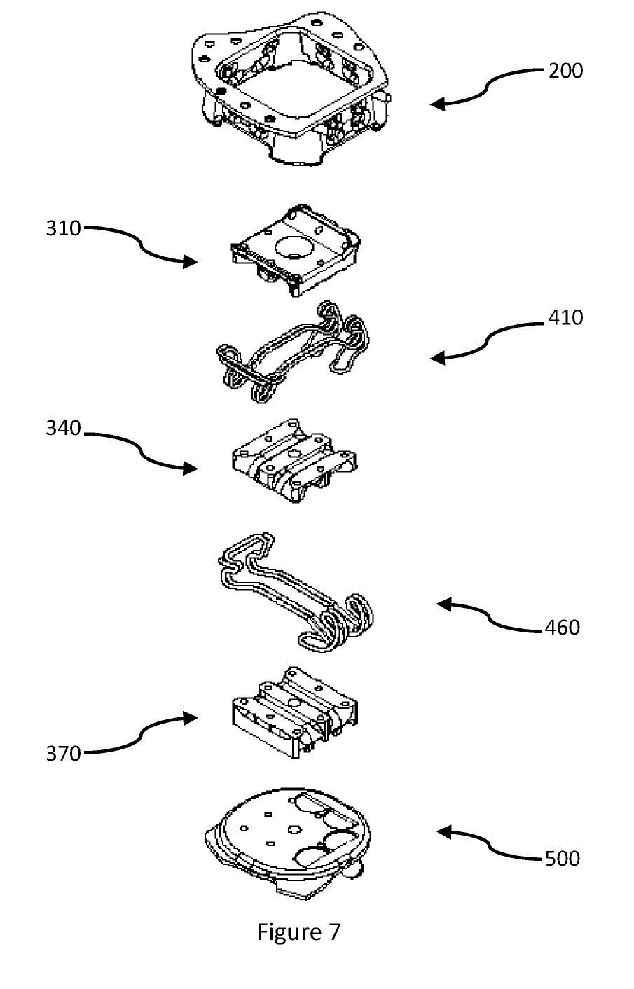

FIG. 7 illustrates an exploded perspective view of an example total ankle replacement in accordance with the disclosure.

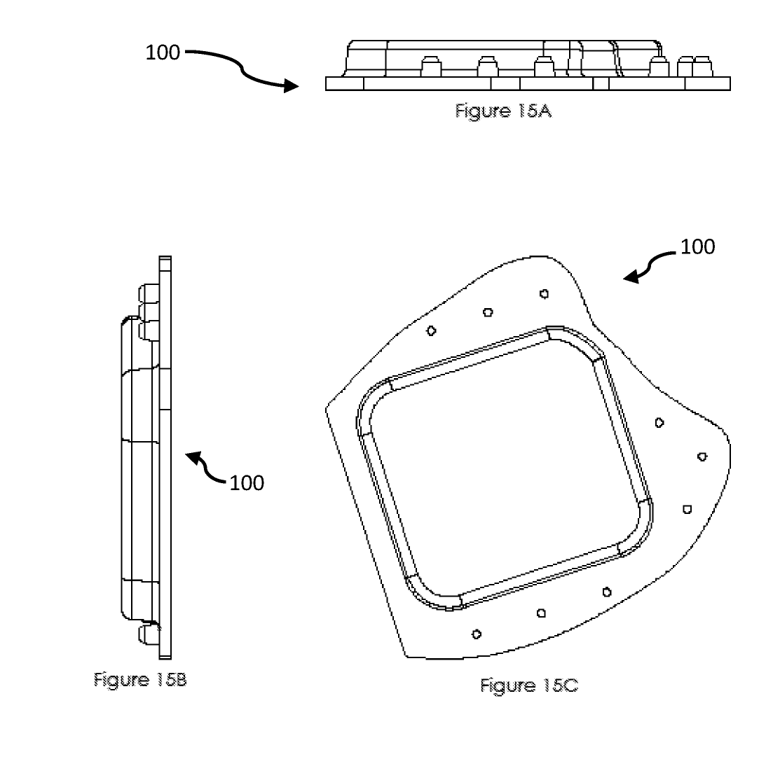

A tibial base plate 100 (FIGS. 15A-C) is configured for coupling between a tibia and tibial component 200.

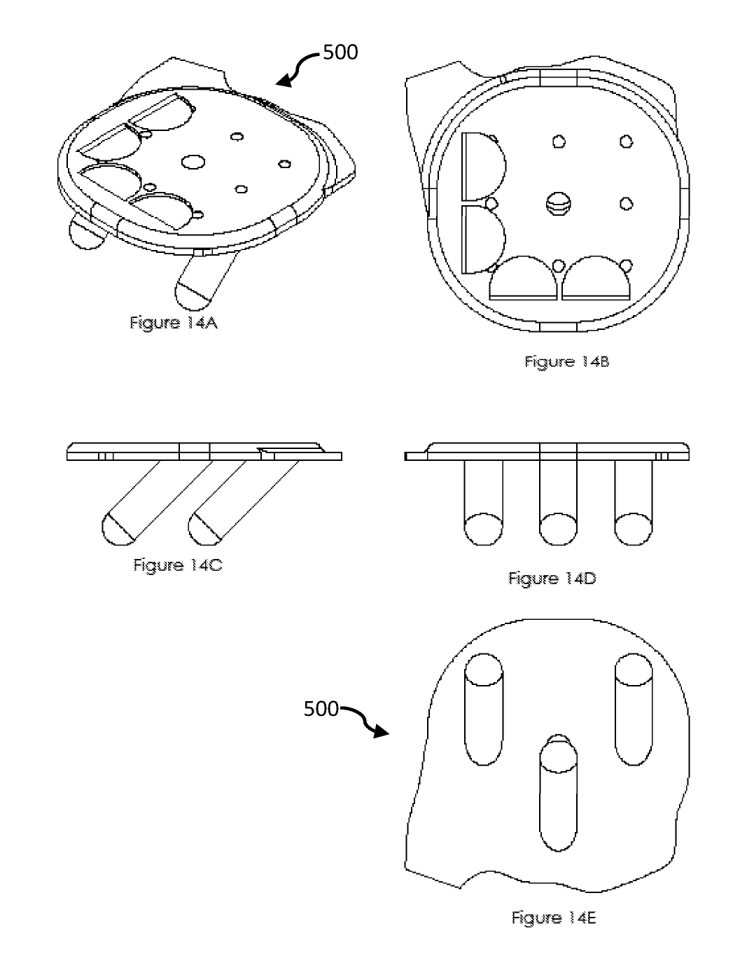

Talar component 300 includes a lower wafer 370, an intermediate wafer 340, and an upper wafer 310 and cooperates with a talar base plate 500 (FIG. 14) configured for coupling between a talus and talar component 300.

Mounting plate 100 and base plate 500 may be custom 3D-printed for a specific patient's anatomy. Optionally, the other parts may be customized for a specific patient as well.

With mounting plate 100 and base plate 500, total ankle replacement joint 10 can be revised by releasing talar wafers 310, 340 and 370 from talar base plate 500, releasing tibial component 200 from tibial base plate 100, then replacing talar and tibial components and the tension members.

Wafers 310, 340 and 370 are coupled together with one or more fasteners. First tension member 410 is clamped between upper 310 and intermediate 340 wafers of talar component 300 such that lengths of the first tension member 410 between tibial component 200 and talar component 300 are fixed against sliding relative to talar component 300. Second tension member 460 is clamped between intermediate wafer 340 of talar component 300 and a lower wafer 370 of talar component 300 such that lengths of second tension member 460 between tibial component 200 and talar component 300 are fixed against sliding relative to talar component 300. Preventing sliding of the tension members 410 and 460 maximizes the life of the cord by minimizing abrasive wear. Optionally, an adhesive may be used in addition to physical clamping force.

Once clamped, the two tension members 410 and 460 may be described as a series of independent segments, each one establishing a specific range of motion between the two compression members. The combined actions of these independent segments enable a complex range of motion in the disclosed replacement joint.

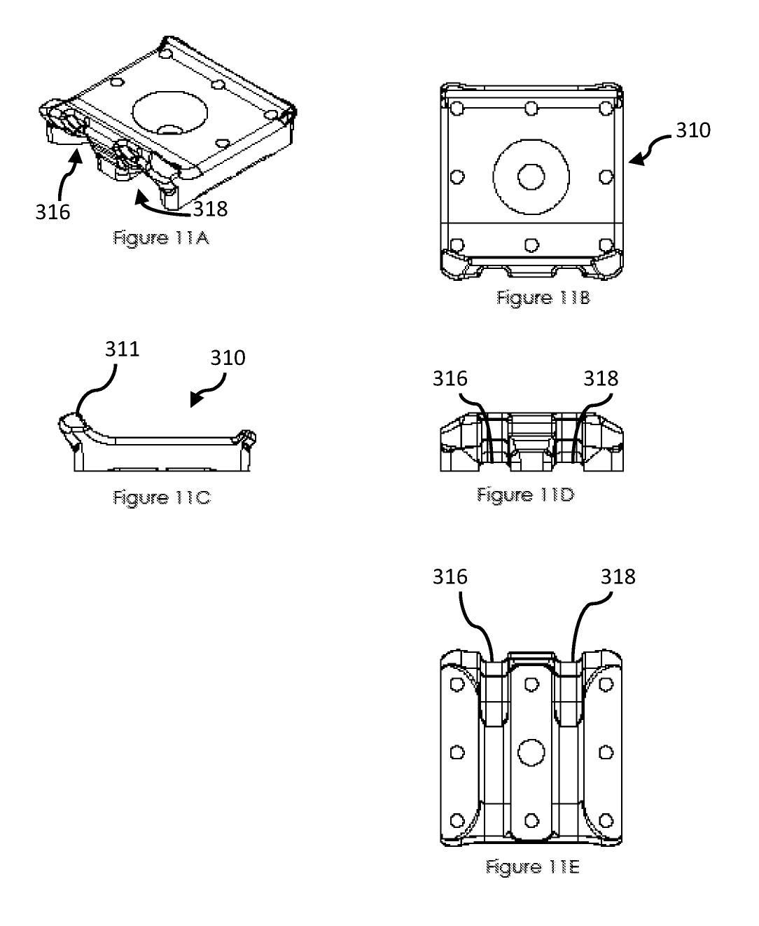

The lengths of each member segment between the clamped talar component and the tibial component may be tailored to tune the “neutral” joint position. These design techniques allow for a very close emulation of the normal human ankle joint's range of motion, despite the tensile nature of this joint not being like the compressive structure of the tibio-talar and talo-fibular joints that it replaces. Further, angles of passageways (formed from mating channels of talar wafers, FIGS. 11-13) through talar component 200 may be adjusted to influence joint range of motion in one direction or another.

In dorsiflexion, a human ankle becomes more stable, owing to the wedge shaped talar trochlea, wider toward the front of the bone. This width spreads the tibia and fibula, stiffening the joint as it moves into dorsiflexion.

The disclosed replacement joint provides stability in dorsiflexion with a tensile based mode. Tibial component 200 establishes a criss-crossed net of tension members, and a talar component 300 clamps onto this net. Considering talar component 300 as a fixed coordinate system, tibial component 200 moves relative to the talar component, by rocking on the web of tension members 410 and 460.

As the tibial component rocks into dorsiflexion, end loops 413 and 418 of the antero-posterior tension member 410 are drawn tight against the two loop-securing anchors posts 376 and 378 on lower wafer 370 of talar component 300, creating an additional constraint between the compression members, and thus additional stability.

Further, when the human ankle moves in dorsiflexion, it also moves into inversion. With medial anteroposterior end loop 418 slightly shorter than lateral anteroposterior end loop 413, an anatomically-analogous motion coupling is created. Alternately, this effect could be achieved with having end loops 418 and 413 the same length, but anchor posts 376 and 378 is modified positions, such that the functional effect was the same.

Similarly, as tibial component 200 rocks into plantarflexion, end loops 463 and 468 of the mediolateral tension member 460 are drawn tight against the two loop-securing anchors or posts 375 and 377 on lower wafer 370 of talar component 300. With posterior mediolateral end loop 468 slightly shorter than anterior mediolateral end loop 463 the disclosed replacement joint 10 moved into eversion during plantarflexion. Also, end loops 463 and 468 serve to limit eversion to a smaller range of motion than inversion, as in the human ankle.

Tibial component 200, talar component 300 and the first 410 and second 460 tension members may be encapsulated within a flexible sheath such as a silicone overmold (not shown) protecting from interaction with the body/immune system/bacterial infection. In an example, the overmold comprises silicone.

The tension members 410 and 460 pass through eyelets 222, 224, 226, 228, 242, 244, 246, 248, 262, 264, 266, 268, 282, 284, 286 and 288 in the tibial component 200, and wrap around portions of the tibial component 200. The friction generated by these wraps, and optionally means for clamping (or adhesively clamping) the wraps if required, restrict the motion of the cord relative to the tibial component, such that it cannot slide.

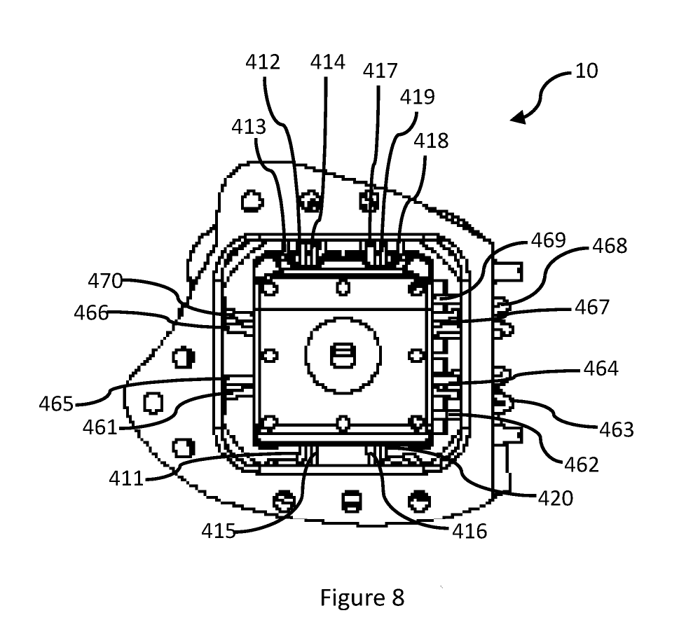

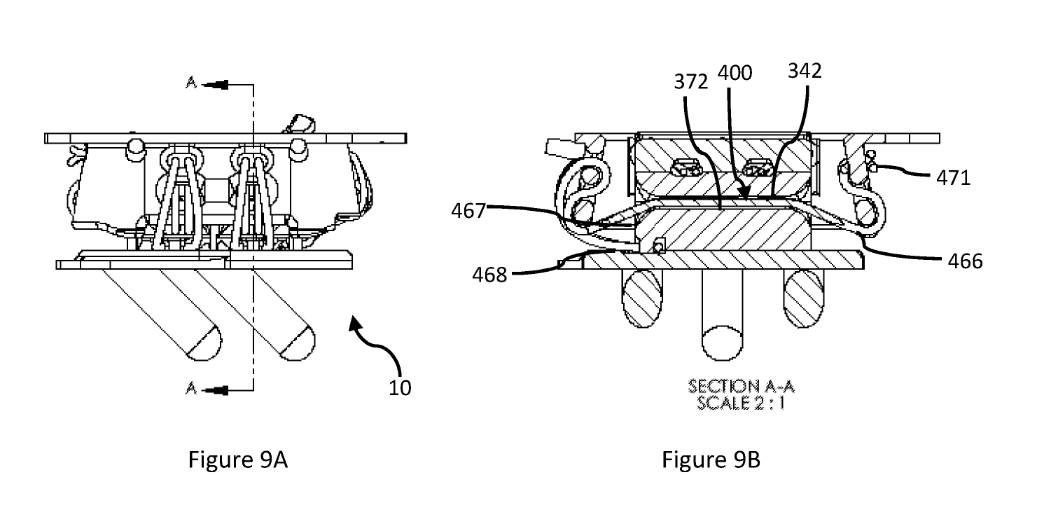

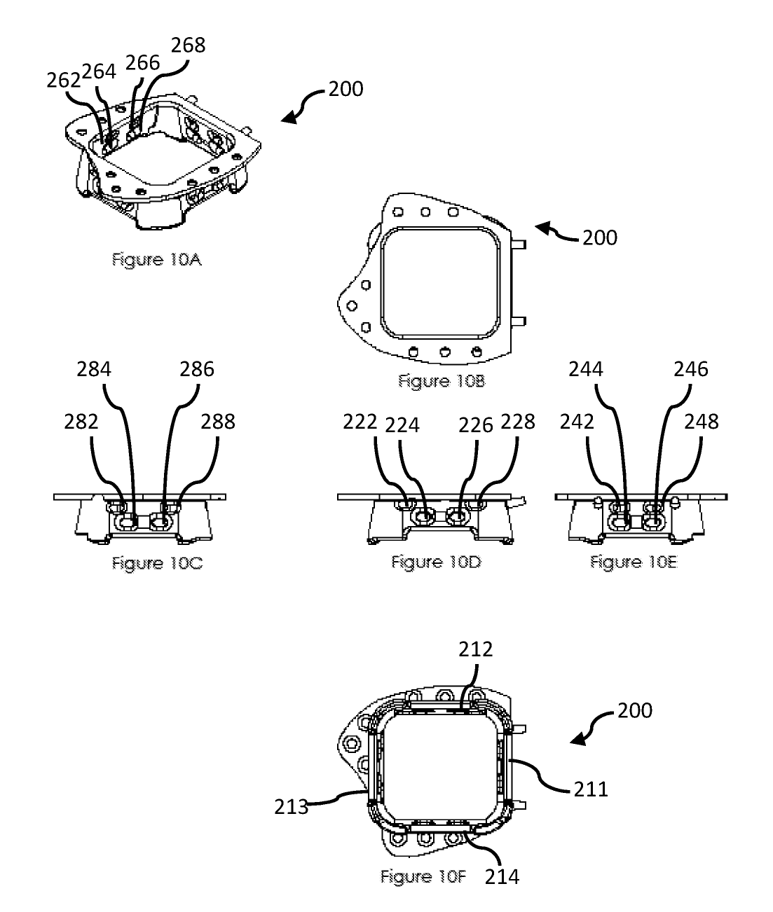

In an example, tension member 410 is oriented anteroposteriorly while tension member 460 is then oriented mediolaterally. The loop pattern is the same between these two tension members, but the length differs between them in specific locations, allowing the mediolateral member to allow and restrict movement differently than the anteroposterior member. Referring to FIGS. 8-10, the paths of first tension member 410 and second tension member 460 are now described.

### Path of AnteroPosterior Tension Member

From an exterior of anterior wall of tibial component 200 first tension member or tendonoid 410 passes through a medial superior eyelet 228 of the anterior wall to an interior of the anterior wall. From medial superior eyelet 228 first tension member 410 passes to and through a medial inferior eyelet 226 of the anterior wall of tibial component 200 from the interior of the anterior wall to the exterior of the anterior wall. This length of first tension member 410 generates friction between tension member 410 and tibial component 200 to prevent relative sliding of the two components.

First tension member 410 extends from medial inferior eyelet 226 of the anterior wall of tibial component 200 under an anterior, inferior edge 212 (or bending sheave) of tibial component 200 and to an anterior wall of talar component 300. This length of first tension member 410 may also be viewed as a segment or web member 420 configured for supporting tibial component 200 relative to talar component 300.

From the anterior wall of talar component 300 first tension member 410 passes through a first anteroposterior passage of talar component 300 to a posterior wall of the talar component 300. This first anteroposterior passage through talar component 300 is formed by the mating of channel 316 of upper wafer 310 with channel 346 of intermediate wafer 340.

First tension member 410 extends from the posterior wall of talar component 300 to and under the posterior inferior edge 214 (or bending sheave) of tibial component 200 to a medial inferior eyelet 266 of the posterior wall of tibial component 200. This length of first tension member 410 may also be viewed as a segment or web member 419 configured for supporting tibial component 200 relative to talar component 300.

From an exterior of the posterior wall of tibial component 200, first tension member 410 passes through medial inferior eyelet 266 to an interior, on to and through a medial superior eyelet 268 of the posterior wall 214 of the tibial component to the exterior. Similar to that described above, this length of first tension member 410 also generates friction between tension member 410 and tibial component 200 to prevent relative sliding of the two components.

First tension member 410 extends from medial superior eyelet 268 of the posterior wall of tibial component 200 and wraps around a first posterior anchor 376 near a posterior, inferior edge of talar component 300.

First tension member then passes back to and through medial superior eyelet 268 of the posterior wall of tibial component 200 to the interior of the posterior wall of the tibial component 200. This length of first tension member 410 may also be viewed as a segment or loop member 418 which is configured to restriction motion. Rotation of talar component 300 relative to tibial component 200 in plantarflexion is limited by loop 418.

Loop member 418, which may be considered a medial loop is shorter than loop member 413 which may be considered a lateral loop.

From medial superior eyelet 268, first tension member 410 passes to and through medial inferior eyelet 266 of the posterior wall of the tibial component from the interior to the exterior.

First tension member 410 passes from medial inferior eyelet 266 of the posterior wall of tibial component 200 under the posterior inferior edge 214 of tibial component 200 to the posterior wall of talar component 300. This length of first tension member 410 may also be viewed as a segment or web member 417 which is configured to support tibial component 200 relative to talar component 300.

From the posterior wall of talar component 300 first tension member 410 passes through first anteroposterior passage of talar component 300 to the anterior wall.

First tension member 410 passes from the anterior wall of talar component 300 to and under the anterior inferior edge 212 of tibial component 200 to the medial inferior eyelet 226 of the anterior wall. This length of first tension member 410 may also be viewed as a segment or web member 416 which is configured to support tibial component 200 relative to talar component 300.

From the exterior of the anterior wall of tibial component 200, tension member 410 passes through medial inferior eyelet 226 to the interior and to and through medial superior eyelet 228 back to the exterior. Similar to that described above, this length of first tension member 410 also generates friction between tension member 410 and tibial component 200 to prevent relative sliding of the two components.

First tension member 410 then passes to and through a lateral superior eyelet 222 of the anterior wall of tibial component 200 from the exterior to the interior, to and through lateral inferior eyelet 224 of the anterior wall of tibial component 200 from the interior of the anterior wall to the exterior. Similar to that described above, this length of first tension member 410 also generates friction between tension member 410 and tibial component 200 to prevent relative sliding of the two components.

First tension member 410 passes from lateral inferior eyelet 224 under the anterior, inferior edge 212 of tibial component 200 and to the anterior wall of talar component 300. This length of first tension member 410 may also be viewed as a segment or web member 415 which is configured to support tibial component 200 relative to talar component 300.

From the anterior wall of the talar component first tension member 410 passes through a second anteroposterior passage of talar component 300 to the posterior wall of talar component 300. This second anteroposterior passage through talar component 300 is formed by the mating of channel 318 of upper wafer 310 with channel 348 of intermediate wafer 340 (FIGS. 11 & 12).

Tension member 410 passes from the posterior wall of talar component 300 to and under the posterior inferior edge of tibial component 200 to a lateral inferior eyelet 264 of the posterior wall of tibial component 200. This length of tension member 410 may also be viewed as segment or web member 414 which is configured to support tibial component 200 relative to talar component 300.

From the exterior of the posterior wall of tibial component 200, first tension member 410 passes through lateral inferior eyelet 264 to the interior and to and through a lateral superior eyelet 262 of the posterior wall from the interior to the exterior. Similar to that described above, this length of first tension member 410 also generates friction between tension member 410 and tibial component 200 to prevent relative sliding of the two components.

First tension member 410 extends from lateral superior eyelet 262 of the posterior wall of tibial component 200 and wraps around a second posterior anchor 378 near the posterior, inferior edge of talar component 300. First tension member 410 then passes to and back through lateral superior eyelet 262 of the posterior wall of tibial component 200 at the exterior. This length of first tension member 410 may also be viewed as a segment or loop member 413 configured to restrict motion. Rotation of talar component 300 relative to tibial component 200 in plantarflexion is limited by loop 413. Loop member 413, which may be considered a lateral loop is shorter than loop member 418 which may be considered a medial loop.

From lateral superior eyelet 262 of the posterior wall of tibial component 200 the first tension member 410 passes to and through lateral inferior eyelet 264 of the posterior wall of tibial component 200 from the interior the posterior wall to the exterior. Similar to that described above, this length of first tension member 410 also generates friction between tension member 410 and tibial component 200 to prevent relative sliding of the two components.

First tension member 410 extends from lateral inferior eyelet 264 of the posterior wall of tibial component 200 under the posterior inferior edge 214 of tibial component 200 to the posterior wall of talar component 300. This length of tension member 410 may also be viewed as a segment or web member 412 configured to support tibial component 200 relative to talar component 300.

The first tension member 410 passes from the posterior wall of talar component 300 through second anteroposterior passage of talar component 300 to the anterior wall.

First tension member 410 extends from the anterior wall of talar component 300 to and under the anterior inferior edge 212 of tibial component 200 to lateral inferior eyelet 224 of the anterior wall of tibial component 200. This length of first tension member 410 may also be viewed as a segment or web member 411 which is configured to support tibial member 200 relative to talar component 300.

From the exterior of the anterior wall of tibial component 200, first tension member 410 passes through lateral inferior eyelet 224 to the interior and then to and through lateral superior eyelet 222 of the anterior wall from the interior to the exterior. Similar to that described above, this length of first tension member 410 also generates friction between tension member 410 and tibial component 200 to prevent relative sliding of the two components. First and second ends of tension member 410 are knotted, clamped, sewn, adhered or otherwise fixedly coupled together at the anterior side of the tibial component.

Generally, first tension member 410 is configured to limit inversion more in plantar flexion than in dorsiflexion.

### Path of Mediolateral Tension Member

Second tension member 460 traverses a similar path to first tension member 410 though generally in a direction angled relative to the path of first tension member 410. For example, second tension member 460 may generally extend in a mediolateral orientation.

From an exterior of a lateral wall of tibial component 200 second tension member 460 passes through anterior superior eyelet 288 to an interior of the lateral wall then to and through anterior inferior eyelet 286 of the lateral wall from the interior to the exterior. This length of second tension member 460 generates friction between tension member 460 and tibial component 200 to prevent relative sliding of the two components.

Second tension member 460 passes from anterior inferior eyelet 286 under a lateral, inferior edge 213 (or bending sheave) of tibial component 200 to a lateral wall of a talar component 300. This length of second tension member 460 may also be viewed as a segment or web member 461 configured to support tibial component 200 relative to talar component 300.

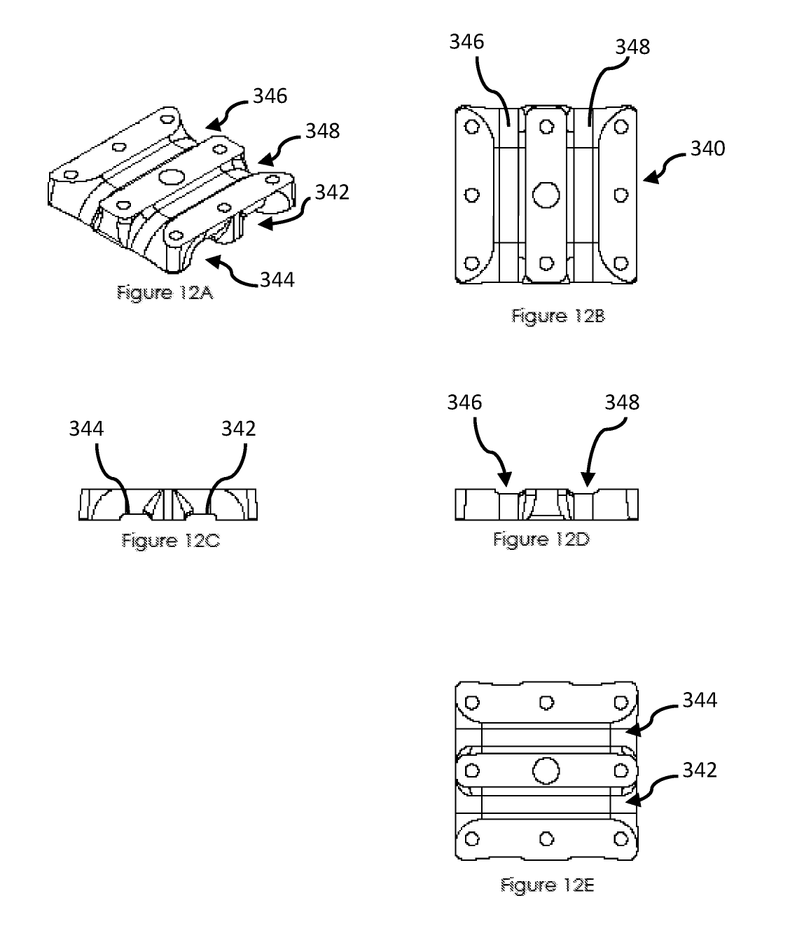

From the lateral wall of talar component 300 second tension member 460 passes through a first mediolateral passage to a medial wall of talar component 300. This first mediolateral passage through talar component 300 is formed by the mating of channel 342 of intermediate wafer 340 with channel 372 of lower wafer 370 (FIGS. 12 & 13).

Second tension member 460 passes from the medial wall of talar component 300 under a medial inferior edge 211 (or bending sheave) of tibial component 200 to anterior inferior eyelet 244 of the medial wall of tibial component 200. This length of second tension member 460 may also be viewed as a segment or web member 462 which is configured to support tibial component 200 relative to talar component 300.

From the exterior of the medial wall of tibial component 200, second tension member 460 passes through anterior inferior eyelet 244 to the interior then to and through anterior superior eyelet 248 from the interior to the exterior. Similar to that described above, this length of second tension member 460 generates friction between tension member 460 and tibial component 200 to prevent relative sliding of the two components.

Second tension member 460 extends from anterior superior eyelet 248 of the medial wall of tibial component 200 and wraps around a first medial anchor 375 near a medial, inferior edge of talar component 300. Second tension member 460 then passes back to anterior superior eyelet 248 at the exterior. This length of second tension member 460 may also be viewed as a segment or loop member 463 which is configured to restrict motion. Rotation of talar component 300 relative to tibial component 200 in eversion is limited by loop member 463. Loop member 463 which may be considered an anterior loop is longer than loop member 468 which may be considered a posterior loop.

From the exterior of the medial wall of tibial component 200, second tension member 460 passes through anterior superior eyelet 248 to the interior then to and through anterior inferior eyelet 246 from the interior to the exterior. Similar to that described above, this length of second tension member 460 also generates friction between tension member 460 and tibial component 200 to prevent relative sliding of the two components.

Second tension member 460 extends from anterior inferior eyelet 246 of the medial wall of tibial component 200 under the medial inferior edge 211 of tibial component 200 to the medial wall of the talar component 300. This length of second tension member 460 may also be viewed as a segment or web member 464 configured to support tibial component 200 relative to talar component 300.

From the medial wall of talar component 300 second tension member 460 passes through the first mediolateral passage of talar component 300 to the lateral wall of talar component 300.

Second tension member 460 passes from the lateral wall of the talar component 300 to and under the lateral inferior edge 213 of tibial component 200 to anterior inferior eyelet 286 of the lateral wall of tibial component 200. This length of second tension member 460 may also be viewed as a segment or web member 465 which is configured to support tibial component 200 relative to talar component 300.

From anterior inferior eyelet 286 of the lateral wall of tibial component 200 second tension member 460 passes from the exterior to the interior, to and through anterior superior eyelet 288 to the exterior, to and through a posterior superior eyelet 282 from the exterior to the interior, to and through a posterior inferior eyelet 284 of the lateral wall of tibial component 200 from the interior to the exterior. Similar to that described above, these lengths of second tension member 460 also generates friction between tension member 460 and tibial component 200 to prevent relative sliding of the two components.

Second tension member 460 passes from posterior inferior eyelet 284 of the lateral wall of tibial component 200 under the lateral, inferior edge 213 of tibial component 200 to the lateral wall of talar component 300. This length of second tension member 460 may also be viewed as a segment or web member 466 which is configured to support tibial component 200 relative to talar component 300.

From the lateral wall of talar component 300 second tension member 460 passes through a second mediolateral passage of talar component 300 to the medial wall of talar component 300. This second mediolateral passage through talar component 300 is formed by the mating of channel 344 of intermediate wafer 340 with channel 374 of lower wafer 370.

Second tension member 460 passes from the medial wall of talar component 300 under the medial inferior edge 211 of tibial component 200 to a posterior inferior eyelet 246 of the medial wall of tibial component 200. This length of second tension member 460 may also be viewed as a segment or web member 467 which is configured to support tibial component 200 relative to talar component 300.

From the exterior of the medial wall of tibial component 200, second tension member 460 passes through posterior inferior eyelet 246 to the interior then to and through a posterior superior eyelet 248 of the medial wall of tibial component 200 to the exterior. Similar to that described above, this length of second tension member 460 also generates friction between tension member 460 and tibial component 200 to prevent relative sliding of the two components.

Second tension member 460 extends from posterior superior eyelet 248 of the medial wall of the tibial component and wraps around a second medial anchor 377 near the medial, inferior edge of the talar component.

Second tension member 460 then passes back to posterior superior eyelet 248 at the exterior. This length of second tension member 460 may also be viewed as a segment or loop member 468 which is configured to restrict motion. Rotation of talar component 300 relative to tibial component 200 in eversion is limited by loop member 468.

Loop member 468 which may be considered a posterior loop is shorter than loop member 463 which may be considered an anterior loop.

From the exterior, second tension member 460 passes through posterior superior eyelet 248 of the medial wall of tibial component 200 to the interior then to and through posterior inferior eyelet 246 of the medial wall of tibial component 200 from the interior to the exterior. Similar to that described above, this length of second tension member 460 also generates friction between tension member 460 and tibial component 200 to prevent relative sliding of the two components.

Second tension member 460 passes from posterior inferior eyelet 246 of the medial wall of tibial component 200 under the medial inferior edge 211 of tibial component 200 to the medial wall of talar component 300. This length of second tension member 460 may also be viewed as a segment or web member 469 which is configured to support tibial component 200 relative to talar component 300.

From the medial wall of talar component 300, second tension member 460 passes through the second mediolateral passage of talar component 300 to the lateral wall of talar component 300.

Second tension member 460 passes from the lateral wall of talar component 300 under the lateral inferior edge 213 of tibial component 200 to posterior inferior eyelet 284 of the lateral wall of tibial component 200. This length of second tension member 460 may also be viewed as a segment or web member 470 which is configured to support tibial component 200 relative talar component 300.

From the exterior second tension member 460 passes through posterior inferior eyelet 284 of the lateral wall of tibial component 200 to the interior then to and through the posterior superior eyelet 282 of the lateral wall of tibial component 200 from the interior to the exterior. Similar to that described above, this length of second tension member 460 also generates friction between tension member 460 and tibial component 200 to prevent relative sliding of the two components. First and second ends of tension member 460 are knotted, sewn, clamped, adhered or otherwise fixedly coupled together at the lateral side of tibial component 200.

Generally, second tension member 460 is configured to limit eversion more in dorsiflexion than in plantarflexion.

Tension members 410 and 460 must have a low enough elongation under load for how closely arranged the compression members are to each other, such that tibial component 200 and talar component 300 are not brought into direct contact within the range of motion of the joint. While other durable materials may be used to provide tension members 410 and 460, plastic fiber cords exhibit a compact form, improved strength over steel, as well as much sharper bending radius and improved fatigue life when bending over small radii. While stronger tension members are generally preferred tension members should be sufficiently flexible to wrap and wind around small bending radii, and bending radii must be selected such that this is achievable. Example materials of construction for tension members 410 and 460 include UHMWPE as well as Dyneema™ and Spectra™ brand rope fibers. In other examples, a more elastic cord may be preferred. In still other examples, a weave of UHMWPE and one or more other elastic elements, or anti-frictional elements may be used. In yet other examples, radio-opaque filaments may be added to the tension members so that damage to the tension members may be discovered by an x-ray.

Referring to FIGS. 11A-E, first, upper, or superior wafer 310 of talar component 300, as described above, includes channels 316 and 318 which cooperate with channels 346 and 348 of intermediate wafer 340 to form anteroposterior passages configured for receiving and clamping a tension member.

Referring to FIGS. 12A-E, second, intermediate or medial wafer 340 of talar component 300, as described above, includes channels 346 and 348 which cooperate with channels 316 and 318 of upper wafer 310 to form anteroposterior passages when the intermediate and superior wafers are mated together. Further, channels 342 and 344 of intermediate wafer 340 cooperate with channels 372 and 374 of lower wafer 370 to form mediolateral passages configured for receiving and clamping a tension member.

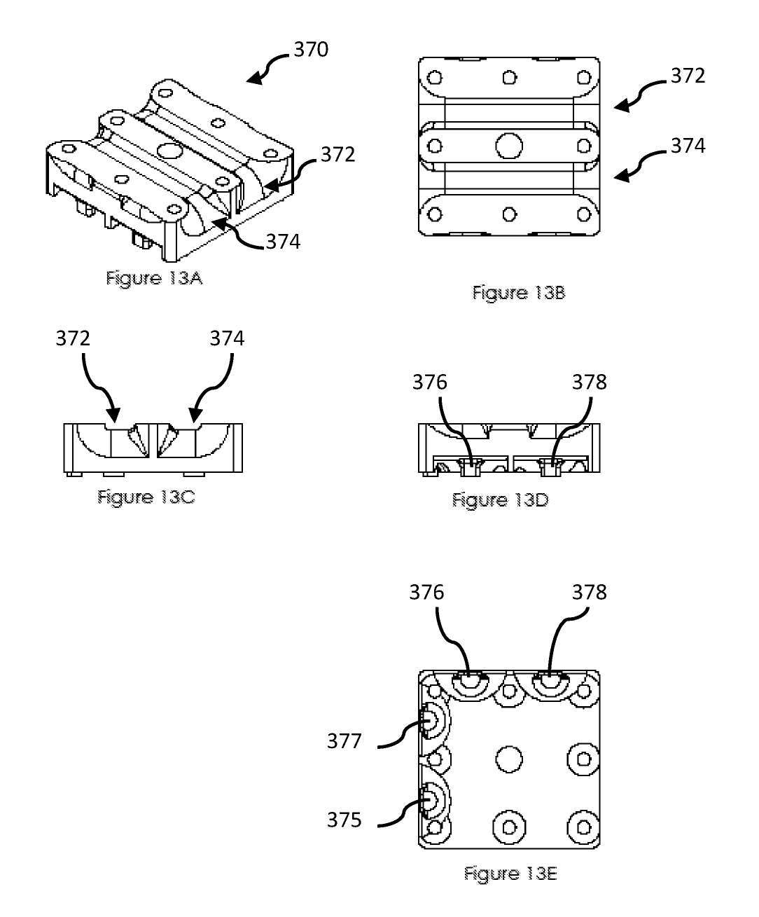

Referring to FIGS. 13A-E, third, lower or inferior wafer of talar component 300, as described above, includes channels 372 and 374 which cooperate with channels 342 and 344 of intermediate wafer 340 to form mediolateral passages when the inferior and intermediate wafers are mated together.

### Simplified Total Ankle Joint

In a simplified form of a total ankle joint in accordance with the disclosure, three fixed-length tension members constrain the motion of the two compression members, relative to each other, in a consistent field of movement, with a virtual center of motion. The two compression members each include first and second peripheral anchors and an intermediate anchor by which the tension members couple the two compression members.

In an example arrangement of simplified implant device for total joint replacement, anterior inferior eyelet 286 functions as a first peripheral anchor of tibial component 200; lateral end of first mediolateral passage of talar component 300, which is formed by the mating of channel 342 of intermediate wafer 340 with channel 372 of lower wafer 370, functions as a first peripheral anchor of talar component 300; medial end of first mediolateral passage of talar component 300 functions as a first intermediate anchor of talar component 300; first medial anchor 375 functions as a second peripheral anchor of talar component 300; anterior superior eyelet 248 functions as a second peripheral anchor of tibial component 200; and anterior inferior eyelet 244 functions as a first intermediate anchor of tibial component 200.

One or more tension members, for example tension member 460, are threaded from the first peripheral anchor 286 of tibial component 200 to the first peripheral anchor (lateral end of the first mediolateral passage formed by channels 342 and 372) of the talar component, from the second peripheral anchor 248 of the tibial component to the second peripheral anchor 375 of the talar component and from the at least one intermediate anchor 244 of the tibial component to the at least one intermediate anchor (medial end of the first mediolateral passage formed by channels 342 and 372) of the talar component. Alternatively, each pair of anchors may be coupled by a separate, individual tension member.

The one or more tension members are configured to support tibial component 200 relative to talar component 300 and to constrain motion of the first peripheral anchor of the tibial component to a fixed radius relative to the first peripheral anchor of the talar component, to constrain motion of the second peripheral anchor of the tibial component about a fixed radius relative to the second peripheral anchor of the talar component and to constrain motion of the at least one intermediate anchor of the tibial component to a fixed radius relative to the intermediate anchor of the talar component.

Motion of the first peripheral anchor of the tibial component relative to the first peripheral anchor of the talar component is constrained to a minor arc of a circle. Motion of the second peripheral anchor of the tibial component relative to the second peripheral anchor of the talar component is constrained to a minor arc of a circle. Motion of the intermediate anchor of the tibial component relative to the intermediate anchor of the talar component is constrained to a minor arc of a circle.

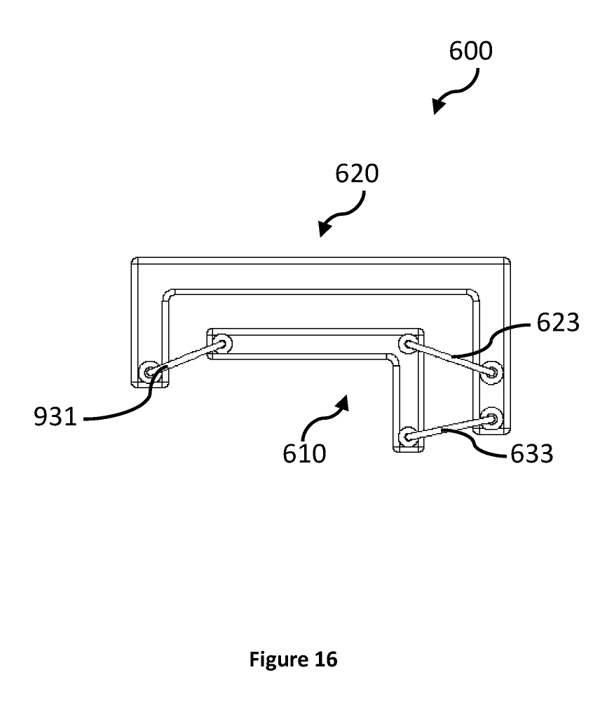

Referring to FIG. 16, in another example arrangement, talar component 610 is coupled with tibial component 620 by a first tension member 931, a second tension member 632 and a third tension member 633.

### Total Ankle Replacement Joint Assembly

A method of assembling a total ankle replacement joint includes coupling a tibial component to a talar component with first and second tension members. First tension member is threaded through the tibial and talar components in a generally anteroposterior orientation and its medial and lateral loops are wound around posterior anchors of the talar component and clamped between first and second wafers of the talar component such that lengths of the first tension member between the tibial component and the talar component are fixed and rotation of the tibial component relative to the talar component in dorsiflexion and plantarflexion are limited.

Second tension member is threaded through the tibial and talar components in a generally mediolateral orientation and its anterior and posterior loops are wound around medial anchors of the talar component and clamped between the second wafer of the talar component and a third wafer of the talar component such that lengths of the first tension member between the tibial component and the talar component are fixed and rotation of the tibial component relative to the talar component in inversion and eversion are limited.

After assembly of the tibial component, the talar component and the first and second tension members, these components are encapsulated within a flexible sheath such as a silicone overmold.

A tibial base plate is coupled with the tibial component during implantation and a talar base plate is coupled with the talar component during implantation.

### Use and Implantation

To use total ankle replacement joint 10, it must be implanted. An incision is made to expose the ankle joint and the inferior end of the tibia and superior end of the talus. A portion of the inferior end of the tibia is resected and tibial base plate 100 (FIGS. 15A-C) is affixed to the resected end of the tibia. Next a portion of the superior surface of the talus is resected and talar base plate 500 (FIGS. 14A-E) is affixed to the resected surface of the talus. With the two base plates 100 and 500 in place, total ankle replacement joint 10 is installed such that tibial component 200 is fixedly coupled with tibial base plate 100 and talar component 300 is fixedly coupled with talar base plate 500.

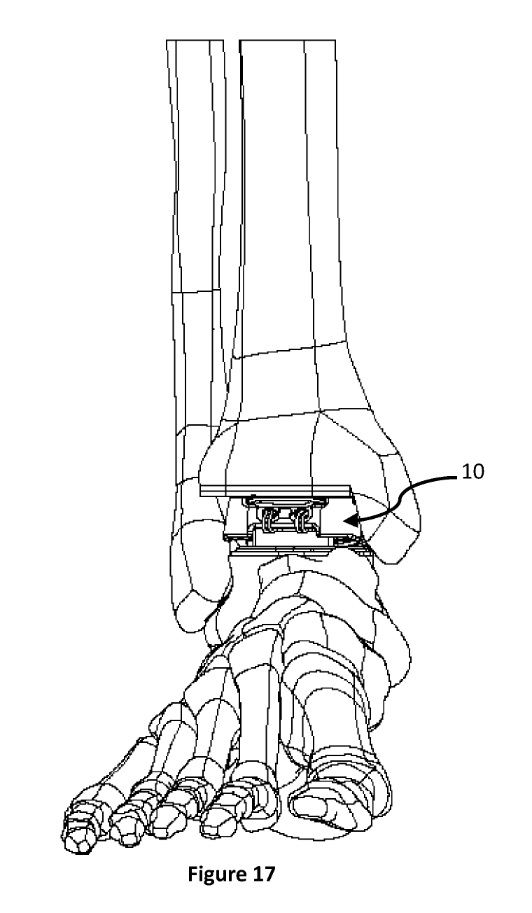

FIG. 17 illustrates a front or anterior view of an example installation of an example total ankle replacement in accordance with the disclosure. Tibial component 200 is supported on talar component 300 by first and second tension members 410 and 460 at four or more points such that contact between the tibial and talar components is substantially prevented.

Any individual total ankle replacement joint 10 may be tuned to meet the appropriate range of motion for the recipient patient. For example, the overall length either or both tension members 410 and 460, the relative lengths of individual segments of both tension members 410 and 460 or both of these may be adjusted to allow for more or less eversion, more or less inversion, more or less dorsiflexion and more or less plantarflexion.

In the event that one or more of tibial component 200, talar component 300, first tension member 410 and second tension member 460 requires repair or replacement, the total ankle joint 10 may be uncoupled from tibial and talar base plates 100 and 500 and removed from the joint space. New tibial and talar components and tension members may then be implanted pre-assembled by coupling with base plates 100 and 500. Disturbing and altering of bone surfaces and the bone/implant interface is avoided and natural bone is preserved.

### Conclusion

The disclosed actions are only illustrative and other alternatives can also be provided where one or more actions are added, one or more actions are removed, or one or more actions are provided in a different sequence without departing from the scope of the claims herein.

Disclosed joints employing two or more compression members and one or more tension members may find further application in other types of implantable joints and compliant joints. With actuators, disclosed joints may be used in robotics applications.

Modifications to embodiments of the present disclosure described in the foregoing are possible without departing from the scope of the present disclosure as defined by the accompanying claims. Expressions such as “including”, “comprising”, “incorporating”, “consisting of”, “have”, “is” used to describe and claim the present disclosure are intended to be construed in a non-exclusive manner, namely allowing for items, components or elements not explicitly described also to be present. Reference to the singular is also to be construed to relate to the plural.

What is claimed is:

1. A total ankle replacement joint, comprising:
a tibial component;
a talar component; and
first and second tension members threaded through the tibial component and the talar component to couple the tibial component with the talar component and support the tibial component relative to the talar component.

2. The total ankle replacement joint as set forth in claim 1, wherein rotation of the talar component relative to the tibial component in eversion is limited by anchoring of loops of the second tension member to a medial side of the talar component.

3. The total ankle replacement joint as set forth in claim 1, wherein rotation of the talar component relative to the tibial component in dorsiflexion is limited by anchoring of loops of the first tension member to a posterior side of the talar component.

4. The total ankle replacement joint as set forth in claim 1, wherein the first and second tension members substantially prevent contact between the tibial and talar components.

5. The total ankle replacement joint as set forth in claim 1, wherein the first tension member is oriented generally anteroposterior, includes lateral and medial loops coupled with posterior anchors of the talar component and wherein the medial loop is shorter than the lateral loop.

6. The total ankle replacement joint as set forth in claim 5, wherein the second tension member is oriented generally mediolateral and includes anterior and posterior loops coupled with medial anchors of the talar component and wherein the posterior loop is shorter than the anterior loop.

7. The total ankle replacement joint as set forth in claim 1, wherein the first tension member is configured to limit inversion more in plantarflexion than in dorsiflexion.

8. The total ankle replacement joint as set forth in claim 1, wherein the second tension member is configured to limit eversion more in dorsiflexion than in plantarflexion.

9. The total ankle replacement joint as set forth in claim 1, wherein the first and second tension members constrain motion of the tibial component relative to the talar component about a moving center.

10. The total ankle replacement joint as set forth in claim 1, wherein the first and second tension members support the tibial component relative to the talar component at four or more points.

11. The total ankle replacement joint as set forth in claim 1, further comprising a tibial base plate configured for coupling between a tibia and the tibial component.

12. The total ankle replacement joint as set forth in claim 1, further comprising a talar base plate configured for coupling between a talus and the talar component.

13. The total ankle replacement joint as set forth in claim 1, wherein where the first and second tension members are threaded through the talar component, the first and second tension members are substantially/approximately perpendicular.

14. The total ankle replacement joint as set forth in claim 1, wherein the tibial component, the talar component and the first and second tension members are encapsulated within a flexible overmold.

15. The total ankle replacement joint as set forth in claim 1, wherein the first tension member is clamped between first and second wafers of the talar component such that lengths of the first tension member between the tibial component and the talar component are fixed.

16. The total ankle replacement joint as set forth in claim 15, wherein the second tension member is clamped between the second wafer of the talar component and a third wafer of the talar component such that lengths of the second tension member between the tibial component and the talar component are fixed.

17. The total ankle replacement joint as set forth in claim 1, wherein the talar component is configured to be received within four walls of the tibial component.

18. An implant device for total joint replacement, the apparatus comprising:
a first implantable articulation component having a first end and a second end, the first end configured for implantation to bone forming a first side of a joint being replaced by the implant device;
a second implantable articulation component having a first end and a second end, the first end configured for implantation to bone forming a second side of the joint being replaced by the implanted device; and
at lease one tension member extending between the first implantable articulation component and the second implantable articulation component, wherein the at least one tension member is configured to dispose the second end of the first implantable articulation component and the second end of the second implantable articulation component toward one another, and the at least one tension member is configured to allow a given amount of articulation of the first implantable articulation component and the second implantable articulation component with respect to one another.

19. An implant device for total joint replacement, comprising:
a tibial component having first and second peripheral anchors and at least one intermediate anchor;
a talar component having first and second peripheral anchors and at least one intermediate anchor;
a first tension member threaded through the first peripheral anchor of the tibial component and the first peripheral anchor of the talar component and configured to constrain motion of the first peripheral anchor of the tibial component to a fixed radius relative to the first peripheral anchor of the talar component;
a second tension member threaded through the second peripheral anchor of the tibial component and the second peripheral anchor of the talar component and configured to constrain motion of the second peripheral anchor of the tibial component about a fixed radius relative to the second peripheral anchor of the talar component; and
a third tension member threaded through the at least one intermediate anchor of the tibial component and the at least one intermediate anchor of the talar component and configured to constrain motion of the at least one intermediate anchor of the tibial component to a fixed radius relative to the intermediate anchor of the talar component.

20. The implant device as set forth in claim 19, wherein:
the first, second and third tension members support the tibial component relative to the talar component and are configured to substantially prevent contact between the tibial component and the talar component.
